(19) United States
(12) Patent Application Publication
(10) Pub. No.: US 2025/0205490 A1
(43) Pub. Date: Jun. 26, 2025

(54) FACILITATING SIGNALS FOR ELECTRICAL STIMULATION

(71) Applicant: Cochlear Limited, Macquarie University, NSW (AU)

(72) Inventors: Christopher Joseph Long, Centennial, CO (US); Joerg Pesch, Mechelen (BE); Sara Ingrid Duran, Denver, CO (US)

(21) Appl. No.: 18/847,409

(22) PCT Filed: Mar. 10, 2023
(86) PCT No.: PCT/IB2023/052315

§ 371 Date: Sep. 16, 2024

Related U.S. Application Data
(60) Provisional application No. 63/320,786, filed on Mar. 17, 2022.

(51) Int. Cl. A61N 1/36; A61N 1/05

(52) U.S. Cl. CPC A61N 1/36038; A61N 1/0541; A61N 1/36128

(57) ABSTRACT

Presented herein are techniques for delivering electrical (current) stimulation to a recipient. More specifically, in accordance with certain embodiments presented herein, at least one facilitating signal having a first spatial extent is delivered to the recipient in order to partially-depolarize nerve cells of the recipient. At least one stimulation signal having a second spatial extent is delivered to the recipient in order to activate at least a subset of the partially-depolarized nerve cells of the recipient. The second spatial extent of the at least one stimulation signal is different from the first spatial extent of the at least one facilitating signal.

### BACKGROUND

### Field of the Invention

The present invention relates generally to use of facilitating signals with electrical stimulation signals.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: delivering at least one facilitating signal having a first spatial extent to partially-depolarize nerve cells of a recipient; and delivering at least one stimulation signal having a second spatial extent to activate at least a subset of the partially-depolarized nerve cells of a recipient, wherein the second spatial extent is different from the first spatial extent.

In another aspect, a method is provided. The method comprises: delivering at least a first facilitating pulse having a first polarity to a nerve cell area using a first electrode configuration associated with a first current spread; and following delivery of the at least first facilitating pulse, delivering at least one biphasic stimulation signal to only at least one portion of the nerve cell area using a second electrode configuration associated with a second current spread that is different from the first current spread.

In another aspect, an implantable medical device is provided. The implantable medical device comprises: one or more input devices configured to receive at least one input signal; one or more processors configured to convert the at least one input signal to at least one output signal; an electrode array configured to be implanted in a recipient; and a stimulator unit configured to generate, based on the at least one output signal, at least one electrical stimulation signal and deliver, via the electrode array, the at least one electrical stimulation signal to a cell area of the recipient, wherein, prior to delivery of the at least one electrical stimulation signal to the cell area, the stimulator unit is configured to generate and deliver, via the electrode array, at least a first facilitating pulse to the cell area, wherein the at least first facilitating pulse is delivered via a first electrode configuration and is configured to only partially-depolarize cells within the cell area.

In another aspect, a medical device is provided. The medical device comprises: one or more processors; and at least one stimulator unit configured to: generate at least a first facilitating pulse having a first polarity, deliver the at least a first facilitating pulse to a cell area of a recipient using a first electrode configuration having an associated first current spread, generate at least one stimulation signal in response to instructions received from the one or more processors, and following delivery of the least a first facilitating pulse, deliver the at least one stimulation signal to the cell area of the recipient using a second electrode configuration having an associated second current spread that is different from the first current spread.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a graph illustrating various phases of an idealized action potential as the potential passes through a nerve cell;

FIG. 3 is a schematic diagram illustrating a facilitating signal and a stimulation signal, in accordance with certain embodiments presented herein;

FIGS. 4A, 4B, and 4C are schematic diagrams illustrating the spatial extent of electrical signals in accordance with certain embodiments presented herein;

FIG. 5A is a schematic diagram illustrating a facilitating signal and a stimulation signal, in accordance with certain embodiments presented herein;

FIG. 5B is another schematic diagram illustrating a facilitating signal and a stimulation signal, in accordance with certain embodiments presented herein;

FIG. 6A is a schematic diagram illustrating a facilitating signal and three stimulation signals, in accordance with certain embodiments presented herein;

FIG. 6B is a schematic diagram illustrating current spread associated with the facilitating signal and the three stimulation signals of FIG. 6A;

FIG. 6C is a schematic diagram illustrating a facilitating signal and two stimulation signals, in accordance with certain embodiments presented herein;

FIG. 6D is a schematic diagram illustrating a facilitating signal and three stimulation signals, in accordance with certain embodiments presented herein;

FIG. 6E is a schematic diagram illustrating a facilitating signal and three stimulation signals, in accordance with certain embodiments presented herein;

FIG. 7 is a flowchart illustrating another example method, in accordance with certain embodiments presented herein;

FIG. 8 is a flowchart illustrating still another example method, in accordance with certain embodiments presented herein;

FIG. 9 is a schematic diagram illustrating a stimulation system with which aspects of the techniques presented herein can be implemented; and

FIG. 10 is a schematic diagram illustrating a vestibular stimulator system with which aspects of the techniques presented herein can be implemented; and

FIG. 11 is a schematic diagram illustrating a retinal prosthesis system with which aspects of the techniques presented herein can be implemented.

### DETAILED DESCRIPTION

Presented herein are techniques for delivering electrical (current) stimulation to a recipient. More specifically, in accordance with certain embodiments presented herein, at least one facilitating signal having a first spatial extent is delivered to the recipient in order to partially-depolarize nerve cells of the recipient. At least one stimulation signal having a second spatial extent is delivered to the recipient in order to activate at least a subset of the partially-depolarized nerve cells of the recipient. The second spatial extent of the at least one stimulation signal is different from (e.g., greater or less than) the first spatial extent of the at least one facilitating signal.

Merely for ease of description, the techniques presented herein are primarily described with reference to a specific hearing device, namely a cochlear implant. It is to be appreciated that the techniques presented herein may also be partially or fully implemented by other types of hearing devices and/or other types of implantable medical devices. For example, the techniques presented herein may be implemented with middle ear auditory prostheses, bone conduction devices, electro-acoustic prostheses, auditory brain stimulators, direct acoustic stimulations, combinations or variations thereof, etc. The techniques presented herein may also be implemented by dedicated tinnitus therapy devices and tinnitus therapy device systems. In further embodiments, the presented herein may also be implemented by, or used in conjunction with, hearables, personal audio devices, in-ear phones, headphones, etc.

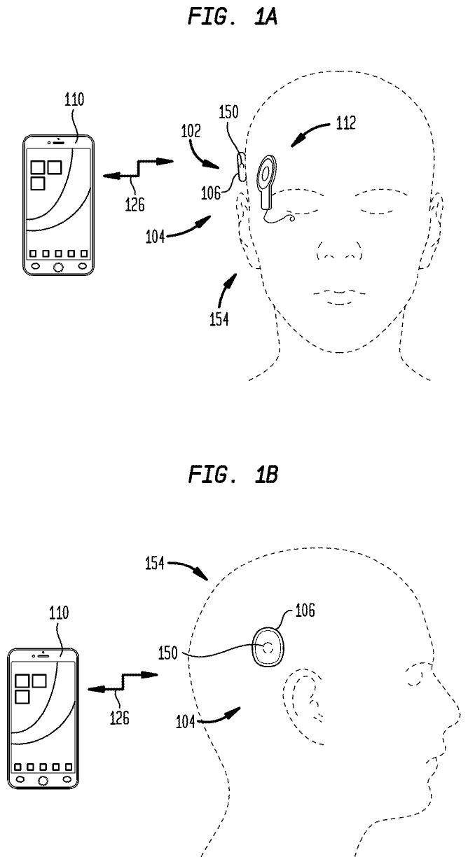

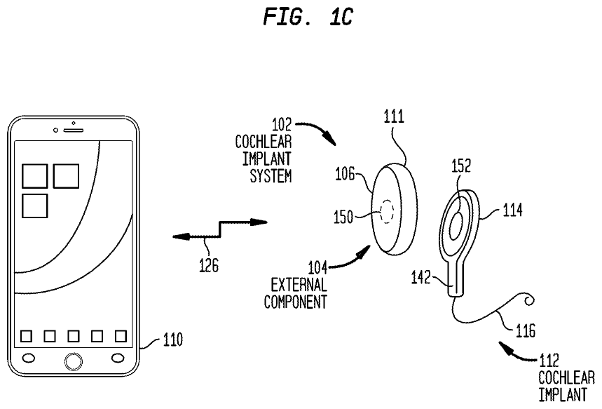

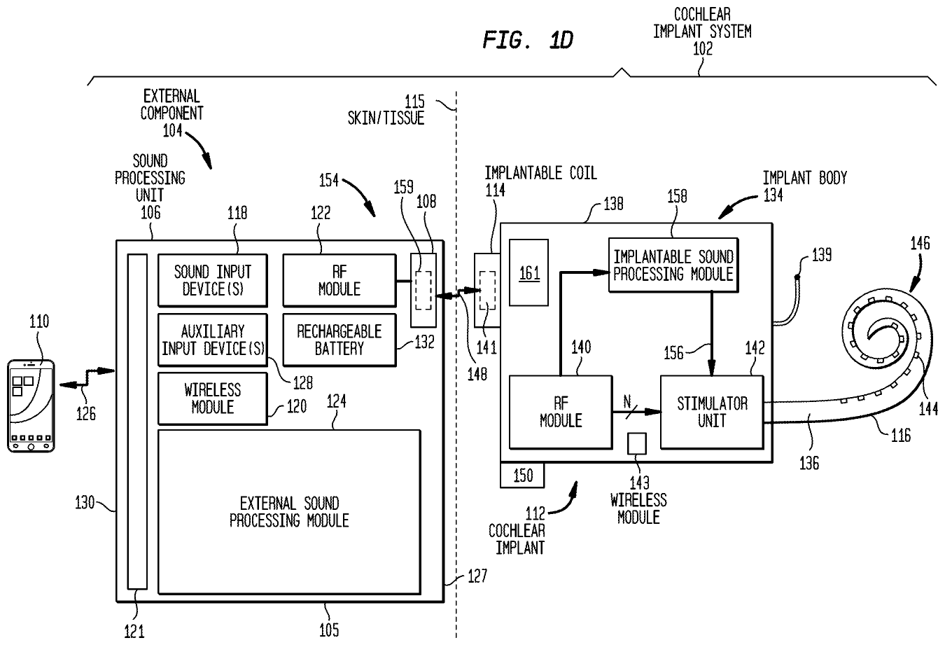

FIGS. 1A-1D illustrate an example cochlear implant system 102 with which aspects of the techniques presented herein can be implemented. The cochlear implant system 102 comprises an external component 104 and an implantable component 112. In the examples of FIGS. 1A-ID, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-ID, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head (e.g., includes an integrated external magnet 159 configured to be magnetically coupled to an implantable magnet 141 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred to as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., environmental signals, such as sound signals, from an ambient environment, data signals, etc.). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter, receiver, and/or transceiver, referred to as a wireless module 120 (e.g., for communication with the external device 110). However, it is to be appreciated that one or more input devices may include additional types of input devices and/or less input devices.

The OTE sound processing unit 106 also comprises the external coil 108, a charging coil 130, a closely-coupled transmitter, receiver, and/or transceiver, referred to as RF module 122, at least one rechargeable battery 132, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in the memory device.

The implantable component 112 comprises an implantable main module (implant body) 134, a lead region 136, and the intracochlear stimulating assembly 116, all configured to be implanted under the skin/tissue (tissue) 115 of the recipient. The implant body 134 generally comprises a hermetically-sealed housing 138 in which an RF module 140 (e.g., an RF receiver, and/or transceiver), a stimulator unit 142, a wireless module 143, an implantable sound processing unit 158, and a rechargeable battery 161 are disposed. The implant body 134 also includes the internal/implantable coil 114 that is generally external to the housing 138, but which is connected to the RF module 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intracochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 159 is fixed relative to the external coil 108 and the implantable magnet 141 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 and the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF module 122, which transcutaneously transfers the output signals (e.g., in an encoded manner) to the implantable component 112 via external coil 108 and implantable coil 114. That is, the output signals are received at the RF module 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, the cochlear implant 112 includes at least an implantable sound sensor arrangement 150 comprising one or more implantable sound sensors (e.g., an implantable microphone and/or an implantable accelerometer).

Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensor 150, potentially in cooperation with one or more other implantable sensors, such as an implantable vibration sensor (not shown in FIGS. 1A-1D), is configured to detect/capture signals (e.g., acoustic sound signals, vibrations, etc.), which are provided to the implantable sound processing module 158. The implantable sound processing module 158 is configured to convert received input signals (received at the implantable sound sensor 150) into electrical signals, sometimes referred to herein as sensed, received, or captured sound signals, for use in stimulating the first ear of a recipient (i.e., the processing module 158 is configured to perform sound processing operations). Stated differently, the one or more processors in implantable sound processing module 158 are configured to execute sound processing logic in memory to convert the received sound signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensor 150 in generating stimulation signals for delivery to the recipient. In other embodiments, the cochlear implant 112 could operate substantially or completely without the external component 104. That is, in such embodiments, the cochlear implant 112 could operate substantially or completely in the invisible hearing mode using the rechargeable battery 161. The rechargeable battery 161 would be recharged via an external charging device.

As noted above, cochlear implant system 102 operates by delivering stimulation signals (current) to the recipient's auditory system, namely the spiral ganglion cells of the recipient's cochlea. In general, the human auditory system is composed of many structural components, some of which are connected extensively by bundles of nerve cells (neurons). Each nerve cell has a cell membrane which acts as a barrier to prevent intercellular fluid from mixing with extracellular fluid. The intercellular and extracellular fluids have different concentrations of ions, which leads to a difference in charge between the fluids. This difference in charge across the cell membrane is referred to herein as the membrane potential (Vm) of the nerve cell. Nerve cells use membrane potentials to transmit signals between different parts of the auditory system.

In nerve cells that are at rest (i.e., not transmitting a nerve signal) the membrane potential is referred to as the resting potential of the nerve cell. Upon receipt of a stimulus, the electrical properties of a nerve cell membrane are subjected to abrupt changes, referred to herein as a nerve action potential, or simply action potential. The action potential represents the transient depolarization and repolarization of the nerve cell membrane. The action potential causes electrical signal transmission along the conductive core (axon) of a nerve cell. Signals may be then transmitted along a group of nerve cells via such propagating action potentials.

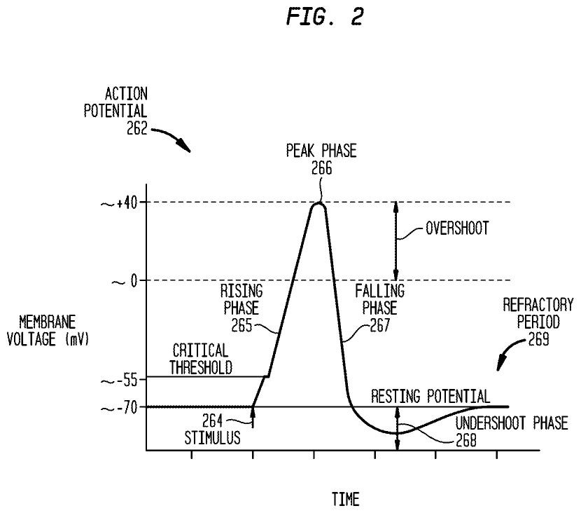

FIG. 2 illustrates various phases of an idealized action potential 262 as the potential passes through a nerve cell. The action potential is presented as membrane voltage in millivolts (mV) versus time. The membrane voltages and times shown in FIG. 2 are for illustration purposes only and the actual values may vary depending on the individual. Prior to application of a stimulus 264 to the nerve cell, the resting potential of the nerve cell is approximately −70 mV. Stimulus 264 is applied at a first time. In normal hearing, this stimulus is provided by movement of the hair cells of the cochlea. Movement of these hair cells results in the release of neurotransmitter into the synaptic cleft, which in return leads to action potentials in individual auditory nerve fibers. In cochlear implants, the stimulus 264 is an electrical stimulation signal (electrical stimulation).

Following application of stimulus 264, the nerve cell begins to depolarize. Depolarization of the nerve cell refers to the fact that the voltage of the cell becomes more positive following stimulus 264. When the membrane of the nerve cell becomes depolarized beyond the cell's critical threshold, the nerve cell undergoes an action potential. This action potential is sometimes referred to as the “firing” or “activation” of the nerve cell. As used herein, the critical threshold of a nerve cell, group of nerve cells, etc. refers to the threshold level at which the nerve cell, group of nerve cells, etc. will undergo an action potential. As used herein, “partial depolarization” or “partially-depolarized” refers cells that have been depolarized to a level that is below/under the cell's critical threshold (e.g., the cells are not activated/fired). In the example illustrated in FIG. 2, the critical threshold level for firing of the nerve cell is approximately −50 mV. The critical threshold and other transitions may be different for various recipients and so the values provided in FIG. 2 are merely illustrative.

The course of the illustrative action potential in the nerve cell can be generally divided into five phases. These five phases are shown in FIG. 2 as a rising phase 265, a peak phase 266, a falling phase 267, an undershoot phase 268, and a refractory phase (period) 269. During rising phase 265, the membrane voltage continues to depolarize and the point at which depolarization ceases is shown as peak phase 2666. In the example of FIG. 2, at this peak phase 266, the membrane voltage reaches a maximum value of approximately 40 mV.

Following peak phase 266, the action potential undergoes falling phase 267. During falling phase 267, the membrane voltage becomes increasingly more negative, sometimes referred to as hyperpolarization of the nerve cell. This hyperpolarization causes the membrane voltage to temporarily become more negatively charged than when the nerve cell is at rest. This phase is referred to as the undershoot phase 268 of action potential 262. Following peak phase 266, there is a time period during which it is impossible or difficult for the nerve cells to fire. This time period is referred to as the refractory phase (period) 269.

As noted above, the nerve cell must obtain a membrane voltage above a critical threshold before the nerve cell may fire/activate. The number of nerve cells that fire in response to electrical stimulation (current) can affect the “spatial extent” of the electrical stimulation. As used herein, the spatial extent of the electrical stimulation (spatial stimulus extent) refers to the amount of acoustic detail (e.g., the spectral detail from the input acoustic sound signal(s)) that is delivered by the electrical stimulation at the implanted electrodes in the cochlea and, in turn, received by the primary auditory neurons (spiral ganglion cells). Stated differently, the spatial extent refers to width along the frequency axis (i.e., along the basilar membrane) of an area of activated nerve cells in response to the delivered stimulation (e.g., the amount of focusing). In general, the spatial extent is proportional to the amount of current spread, meaning a greater spatial extent is associated with greater current spread, while lower spatial extent is associated with lower current spread.

There are different techniques for controlling the spatial extent of the electrical stimulation signals (e.g., to control the width along the frequency axis of an area of activated nerve cells in response to delivered stimulation). For example, the spatial extent of electrical stimulation may be controlled, for example, through the use of different electrode configurations for given stimulation channels to activate nerve cell regions of different widths. Monopolar stimulation, for instance, is an electrode configuration where for a given stimulation channel the current is “sourced” via one of the intra-cochlea electrodes 144, but the current is “sunk” by an electrode outside of the cochlea, sometimes referred to as the extra-cochlear electrode (ECE) 139 (FIG. 1D). Monopolar stimulation typically exhibits a large degree of current spread (i.e., wide stimulation pattern) and, accordingly, has a large spatial extent.

Other types of electrode configurations, such as tripolar, focused multi-polar (FMP), a.k.a. “phased-array” stimulation, etc. typically reduce the size of an excited neural population by “sourcing” the current via one or more of the intra-cochlear electrodes 144, while also “sinking” the current via one or more other proximate intra-cochlear electrodes. Bipolar, tripolar, focused multi-polar and other types of electrode configurations that both source and sink current via intra-cochlear electrodes are generally and collectively referred to herein as “focused” or “multipolar” stimulation. Focused stimulation typically exhibits a smaller degree of current spread (i.e., narrow stimulation pattern) when compared to monopolar stimulation and, accordingly, has a lower spatial extent than monopolar stimulation. Likewise, other types of electrode configurations, such as double electrode mode, and wide channels typically increase the size of an excited neural population by “sourcing” the current via multiple neighboring intra-cochlear electrodes.

Still other types of electrode configurations, sometimes referred to herein as, focused or multipolar stimulation with partial far-field return, operate by sourcing the current via one or more of the intra-cochlear electrodes 144, while also sinking the current via one or more other proximate intra-cochlear electrodes an the ECE 139. That is, in focused or multipolar stimulation with partial far-field return, the return current path is at least partially through the ECE 139, which is different from full intracochlear multipolar stimulation where the current is only sunk via the intra-cochlear electrodes 144. Multipolar stimulation with partial far-field return exhibits a degree of current spread that is less than monopolar stimulation, but larger than that of full intracochlear multipolar stimulation.

The cochlea is tonotopically mapped, that is, partitioned into regions each responsive to sound signals in a particular frequency range. In general, the basal region of the cochlea is responsive to higher frequency sounds, while the more apical regions of the cochlea are responsive to lower frequencies. The tonotopic nature of the cochlea is leveraged in cochlear implants such that specific acoustic frequencies are allocated to the electrodes 144 of the stimulating assembly 116 that are positioned close to the corresponding tonotopic region of the cochlea (i.e., the region of the cochlea that would naturally be stimulated in acoustic hearing by the acoustic frequency). That is, in a cochlear implant, specific frequency bands are each mapped to a set of one or more electrodes that are used to stimulate a selected (target) population of cochlea nerve cells. The frequency bands and associated electrodes form a stimulation channel that delivers stimulation signals to the recipient.

In general, it is desirable for a stimulation channel to stimulate only a narrow region of neurons such that the resulting neural responses from neighboring stimulation channels have minimal overlap. Accordingly, the ideal stimulation strategy in a cochlear implant would use focused stimulation/lower spatial extent to evoke perception of all sound signals at any given time. Such a strategy would, ideally, enable each stimulation channel to stimulate a discrete tonotopic region of the cochlea to better mimic natural hearing and enable better perception of the details of the sound signals. However, although focused stimulation/lower spatial extent generally improves hearing performance, this improved hearing performance comes at the expense of higher levels of stimulation current. That is, there is a trade-off between stimulation power and amount of focusing/spatial extent. For example, it has been discovered that the current needed for certain types of focused stimulation (e.g., to cause the target nerve cells to fire) can be, in certain arrangements, sixty (60) current levels (CLs) higher than the current needed in monopolar stimulation, which equates to nearly two doublings in current for focused stimulation relative to monopolar stimulation. Presented herein are techniques to achieve the benefits of focused stimulation with lower overall current levels and, as such, lower overall power consumption by delivering/applying facilitating signals and stimulation signals having different spatial extents. For example, in certain embodiments, a relatively less focused (relatively greater spatial extent) facilitating signal is delivered before or around a relatively more focused (relatively lower spatial extent) stimulation signal. Such arrangements may result in a use of lower current levels, while still achieving the benefits of focused stimulation (e.g., relatively narrower current spread).

More specifically, in accordance with embodiments presented herein, at least one facilitating signal having a first spatial extent is used to partially-depolarize a recipient's nerve cells (e.g., depolarize only to a level that is below the critical threshold of the nerve cells). Thereafter, at least one stimulation signal having a second spatial extent is delivered to the partially-depolarized nerve cells to cause at least a subset of the partially-depolarized nerve cells to undergo an action potential and, accordingly, activate/fire. In certain embodiments presented herein, the second spatial extent of the at least one stimulation signal is less than (i.e., more focused, less current spread), than the first spatial extent. Stated differently, in these embodiments, at least one facilitating signal having a relatively larger degree of current spread (i.e., stimulation current with a greater spatial extent) is delivered to “prime” or “charge” a larger area of nerve cells to a sub-firing threshold, then at least one stimulation signal having a relatively smaller degree of current spread (i.e., stimulation current with a relatively lower spatial extent) is delivered to fire only a subset or relatively smaller number of the primed nerve cells. This specific combination of at least one less-focused facilitating signal followed by at least one relatively more focused stimulation signal may achieve the benefits of traditional focused stimulation, with the need for less stimulation current.

In other embodiments presented herein, second spatial extent of the at least one stimulation signal is greater (i.e., less focused, greater current spread) than first spatial extent of the facilitating signal. Stated differently, at least one facilitating signal having a relatively smaller degree of current spread (i.e., stimulation current with a low spatial extent) is delivered to “prime” or “charge” a smaller area of nerve cells to a sub-firing threshold, then at least one stimulation signal having a relatively larger degree of current spread (i.e., stimulation current with a relatively larger spatial extent) is delivered to fire at least a subset of the primed nerve cells. This specific combination of at least one more-focused facilitating signal followed by at least one relatively less focused stimulation signal may achieve the benefits of traditional focused stimulation, with the need for less stimulation current.

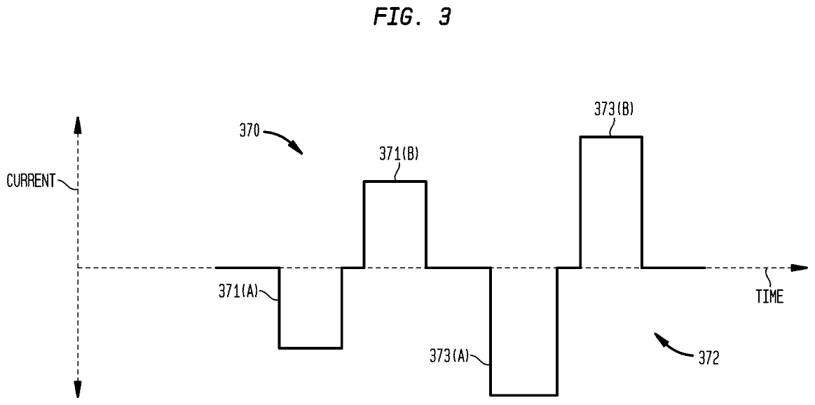

FIG. 3 is a schematic diagram illustrating use of a facilitating signal, in accordance with aspects of the techniques presented herein. More specifically, FIG. 3 illustrates that a first biphasic facilitating signal 370 having a first spatial extent is delivered to the nerves. In particular, as shown in FIG. 3, the biphasic facilitating signal 370 comprises a first facilitating pulse 371(A) having a first polarity, and a second facilitating pulse 371(B) having a second polarity that is opposite to the first polarity. The biphasic facilitating signal 370 is delivered to the nerve cells (e.g., with an intensity/level, timing, etc.) to depolarize the nerve cells only to a level that is below the critical threshold of the nerve cells (e.g., partially-depolarize the nerve cells). That is, the biphasic facilitating signal 370 is configured such that the nerve cells will not fire (e.g., will not undergo an action potential) in response to only the biphasic facilitating signal 370.

After delivery of the biphasic facilitating signal 370, a biphasic stimulation signal 372 having a second spatial extent is delivered to the only a subset of the nerve cells depolarized by the biphasic facilitating signal 370. As shown in FIG. 3, the biphasic stimulation signal 372 comprises a first stimulation pulse 373(A) having a first polarity, and a second stimulation pulse 373(B) having a second polarity that is opposite to the first polarity. The biphasic stimulation signal 372 is delivered to the nerve cells (e.g., with an intensity/level, timing, etc.) to cause the subset of the nerve cells to fire (e.g., undergo an action potential). This firing of the subset of the nerve cells causes a percept by the recipient. In the case of cochlear implants, this percept is the perception of sound.

As noted above, in the example of FIG. 3, the biphasic facilitating signal 370 has a first spatial extent and the biphasic stimulation signal 372 has a second spatial extent. In one example, the second spatial extent of the biphasic stimulation signal 372 is less (i.e., has less current spread) than the first spatial extent of the biphasic facilitating signal 370. That is, in accordance with certain embodiments, the biphasic facilitating signal 370 has a relatively wider current spread than the current spread of the biphasic stimulation signal 372. The relative differences in these spatial extents can be the result of different electrode configurations, such as monopolar stimulation, wide/defocused stimulation, focused (e.g., multipolar current focusing) stimulation, etc.

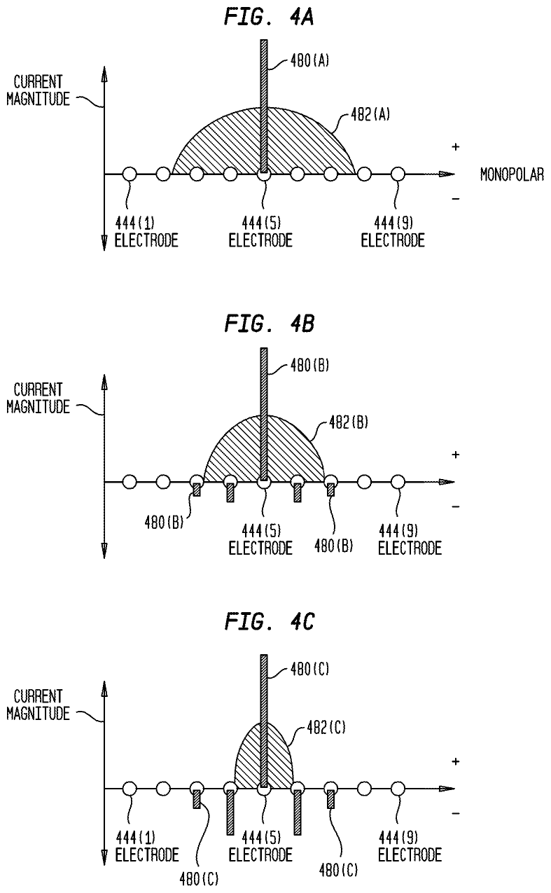

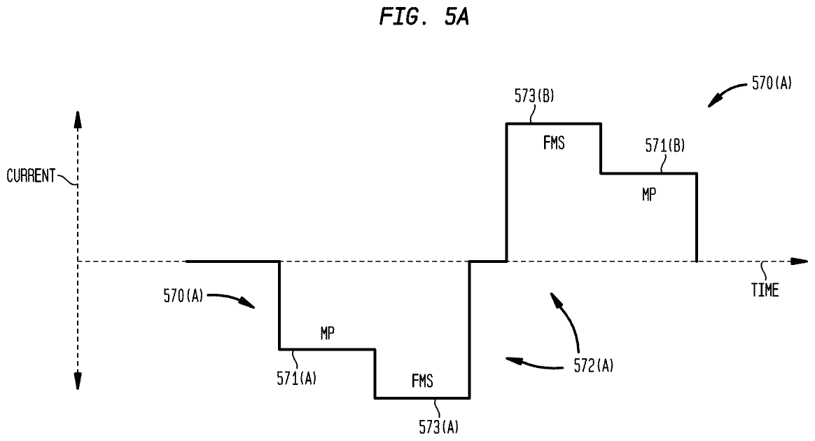

For example, FIGS. 4A, 4B, and 4Care schematic diagrams illustrating exemplary electrode currents and spatial extents for different channel configurations. It is to be appreciated that the spatial extents shown in FIGS. 4A-4C are generally illustrative and that, in practice, the stimulation current may spread differently in different recipients.

Each of the FIGS. 4A-4C illustrates a plurality of electrodes shown as electrodes 444(1)-444(9), which are spaced along the recipient's cochlea frequency axis (i.e., along the basilar membrane). FIGS. 4A-4C also include solid lines of varying lengths that extend from various electrodes to generally illustrate the intra-cochlear stimulation current 480(A)-480(E) delivered in accordance with a particular channel configuration. However, it is to be appreciated that, in these embodiments, the stimulation is delivered to a recipient using charge-balanced waveforms, such as biphasic current pulses and that the length of the solid lines extending from the electrodes in each of FIGS. 4A-4C illustrates the relative “weights” that are applied to both phases of the charge-balanced waveform at the corresponding electrode in accordance with different channel configurations, but with different polarities in each phase. As described further below, the different stimulation currents 480(A) and480(C) (i.e., different channel weightings) results in different stimulation patterns 482(A) and482(C), respectively, of voltage and neural excitation along the frequency axis of the cochlea.

Referring first to FIG. 4A, shown is the use of a monopolar channel configuration where all of the intra-cochlear stimulation current 480(A) is delivered with the same polarity via a single electrode 444(5). In this embodiment, the stimulation current 480(A) is sunk by an extra-cochlear return contact which, for ease of illustration, has been omitted from FIG. 4A. The intra-cochlear current 480(A) generates a current spread pattern 482(A) where, as shown, the current spreads across neighboring electrodes 444(3), 444(4), 444(6), and 444(7). The current spread pattern 482(A) represents the spatial extent of the monopolar channel configuration.

FIGS. 4B and 4C illustrate focused channel configuration where intracochlear compensation currents are added to decrease the spread of current along the frequency axis of the cochlea. The compensation currents are delivered with a polarity that is opposite to that of a primary/main current. In general the more compensation current at nearby electrodes, the more focused the resulting stimulation pattern (i.e., the lower the width of the current spread pattern and thus increasingly lower spatial extents). That is, the spatial extent is reduced by introducing increasing large compensation currents on electrodes surrounding the central electrode with the positive current.

More specifically, in FIG. 4B current 480(B) of a first polarity is delivered via electrode 444(5) and current 480(B) of an opposite polarity is delivered via the neighboring electrodes, namely electrodes 444(3), 444(4), 444(6), and 444(7). The intra-cochlear current 480(B) generates a current spread pattern 482(B) where, as shown, the current only spreads across electrodes 444(4)-444(6). In FIG. 4C, current 480(C) of a first polarity is delivered via electrode 444(5), while current 480(C) of the opposite polarity is delivered via the neighboring electrodes, namely electrodes 444(3), 444(4), 444(6), and 444(7). The intra-cochlear current 480(C) generates a current spread pattern 482(C) which, as shown, is generally localized to the spatial area adjacent electrode 444(5).

The difference in the current spread patterns 482(B) and 482(C) in FIGS. 4B and 4C, respectively, is due to the magnitudes (i.e., weighting) of opposite polarity current delivered via the neighboring electrodes 444(3), 444(4), 444(6), and 444(7). In particular, FIG. 4B illustrates a partially focused configuration where the compensation currents do not fully cancel out the main current on the central electrode and the remaining current goes to a far-field extracochlear electrode (not shown). This is an example of stimulation using a partial far-field return (e.g., multipolar stimulation with a partial far-field return). FIG. 4C is a fully focused configuration where the compensation currents fully cancel out the main current on the central electrode 444(5) (i.e., no far-field extracochlear electrode is used).

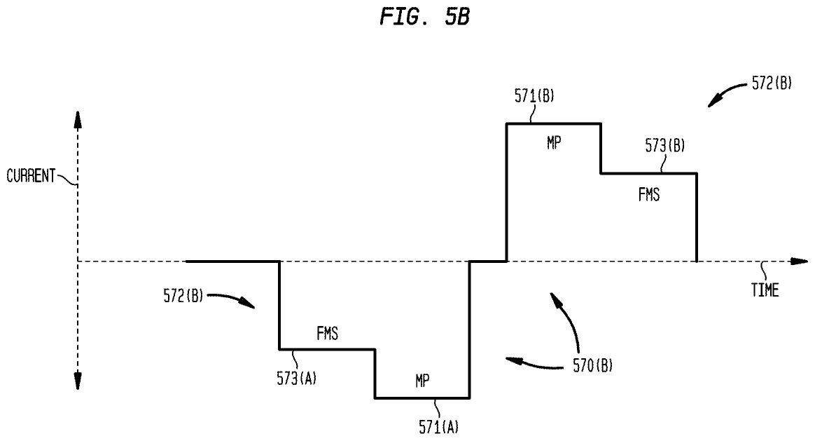

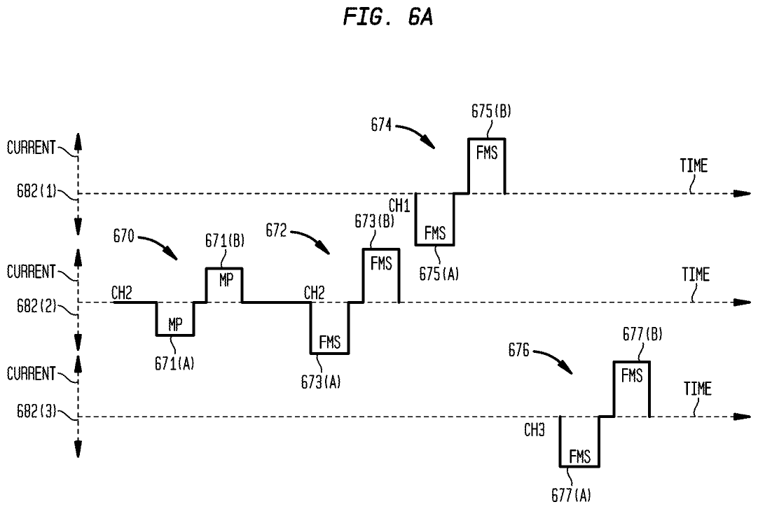

As noted, FIG. 3 illustrates an embodiment in which a biphasic signal is used as a facilitating signal to partially depolarize nerve cells prior to delivery of a biphasic stimulation signal to cause a subset of the partially depolarized nerve cells to fire (e.g., a monopolar biphasic conditioning signal followed by a more focused biphasic stimulation signal or a more focused biphasic conditioning signal followed by a monopolar biphasic stimulation signal). In the embodiment of FIG. 3, the charge associated with the facilitating signal is collected before delivery of the stimulation signal. FIG. 5A illustrates an alternative embodiment in which the charge associated with the facilitating signal is not collected until after delivery of the stimulation signal. That is, FIG. 5A illustrates an embodiment in which a stimulation signal splits a facilitating signal (e.g., a split monopolar facilitating signal with an inter-posed multipolar stimulation signal).

More specifically, FIG. 5A illustrates a facilitating signal 570(A) that is comprised of a first monophasic facilitating pulse 571(A) having a first polarity and a second facilitating monophasic pulse 571(B) having a second polarity that is opposite the first polarity. FIG. 5A also illustrates a biphasic stimulation signal 572(A) comprised of a first stimulation pulse 573(A) having a first polarity, and a second stimulation pulse 573(B) having a second polarity that is opposite to the first polarity. As shown, the biphasic stimulation signal 572(A) (i.e., stimulation pulses 573(A) and 573(B)) is delivered between the facilitating pulses 571(A) and 571(B) of the facilitating signal 570(A). That is, in this embodiment, the facilitating signal 570(A) surrounds the stimulation signal 572(A).

In accordance with the embodiments of FIG. 5A, the monophasic facilitating pulses 571(A) and 571(B) of the facilitating signal 570(A) are delivered to the nerves using an electrode configuration having an associated first spatial extent, while the stimulation pulses 573(A) and 573(B) of the stimulation signal 572(A) are delivered to the nerves using an electrode configuration having an associated second spatial extent that is less than the first spatial extent. Stated differently, the stimulation pulses 573(A) and 573(B) are configured so as to result in a current spread that is less than the current spread resulting from the facilitating pulses 571(A) and 571(B).

In the example of FIG. 5A, the facilitating pulse 571(A) is delivered to the nerve cells (e.g., with an intensity/level, timing, etc.) to depolarize the nerve cells only to a level that is below the critical threshold of the nerve cells. That is, the facilitating pulse 571(A) is configured such that the nerve cells will not fire (e.g., will not undergo an action potential) in response to only the facilitating pulse 571(A).

After delivery of the facilitating pulse 571(A), the biphasic stimulation signal 572(A) (having the second spatial extent) is delivered to the only a subset of the nerve cells depolarized by the facilitating pulse 571(A). The biphasic stimulation signal 572(A) is delivered to the nerve cells (e.g., with an intensity/level, timing, etc.) to cause the subset of the nerve cells to fire (e.g., undergo an action potential). This firing of the subset of the nerve cells causes a percept by the recipient. In the case of cochlear implants, this percept is the perception of sound. After delivery of the biphasic stimulation signal 572(A), the facilitating pulse 571(B) is delivered to balance the charge in the nerve cells (e.g., to remove the charge introduced by facilitating pulse 571(A)).

As noted above, in the example of FIG. 5A, the pulses 571(A) and 571(B) have a first spatial extent and the stimulation pulses 573(A) and 573(B) have a second spatial extent that is less than the first spatial extent (i.e., less current spread). That is, in accordance with these embodiments, the facilitating signal 570(A) has a relatively wider current spread than the current spread of the biphasic stimulation signal 572(A). The relative differences in these spatial extents can be the result of different electrode configurations, such as monopolar stimulation, wide/defocused stimulation, focused (e.g., multipolar current focusing) stimulation, etc., as described, for example, with reference to FIGS. 4A-4C.

FIG. 5B illustrates a facilitating signal 570(B) that is comprised of a first monophasic facilitating pulse 571(A) having a first polarity and a second facilitating monophasic pulse 571(B) having a second polarity that is opposite the first polarity. FIG. 5B also illustrates a biphasic stimulation signal 572(B) comprised of a first stimulation pulse 573(A) having a first polarity, and a second stimulation pulse 573(B) having a second polarity that is opposite to the first polarity. As shown, the biphasic facilitating signal 570(B) (i.e., facilitating pulses 571(A) and 571(B)) is delivered between the stimulation pulses 573(A) and 573(B) of the stimulation signal 572(B). That is, in this embodiment, the stimulation signal 572(B) surrounds the facilitating signal 570(B).

In accordance with the embodiments of FIG. 5B, the facilitating pulses 571(A) and 571(B) of the facilitating signal 570(B) are delivered to the nerves using an electrode configuration having an associated first spatial extent, while the stimulation pulses 573(A) and 573(B) of the stimulation signal 572(B) are delivered to the nerves using an electrode configuration having an associated second spatial extent that is less than the first spatial extent. Stated differently, the stimulation pulses 573(A) and 573(B) are configured so as to result in a current spread that is less than the current spread resulting from the facilitating pulses 571(A) and 571(B).

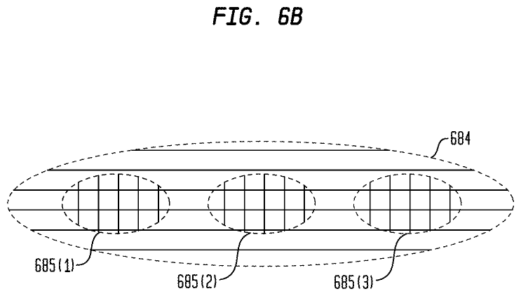

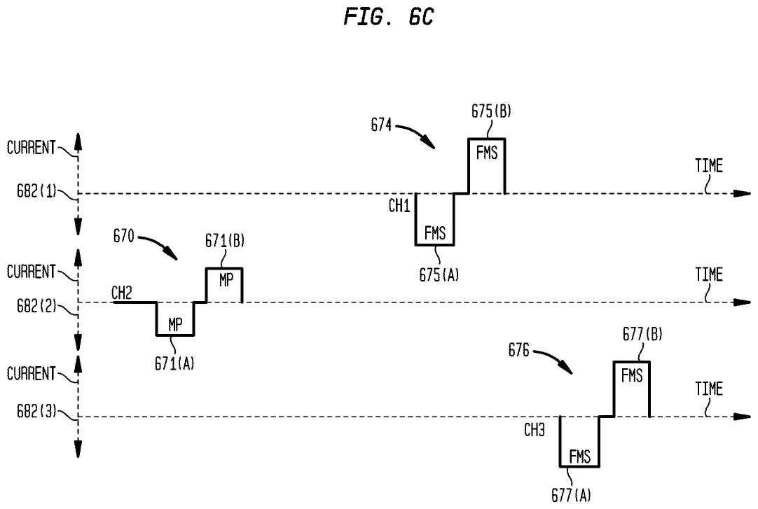

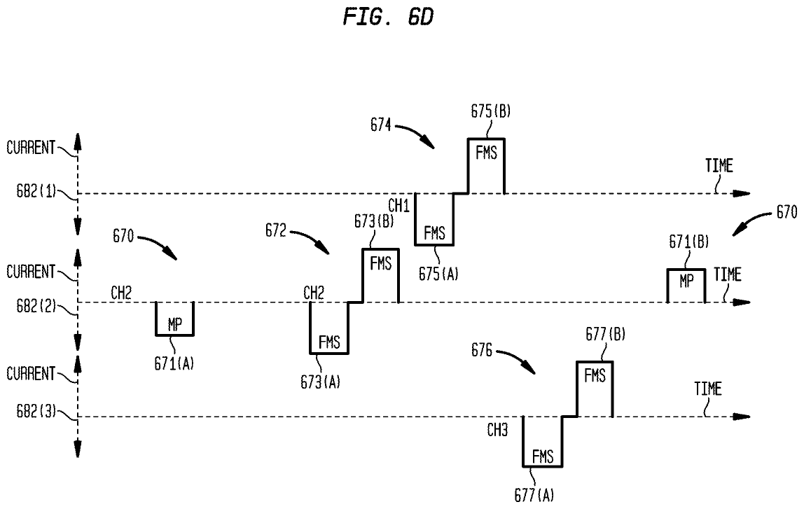

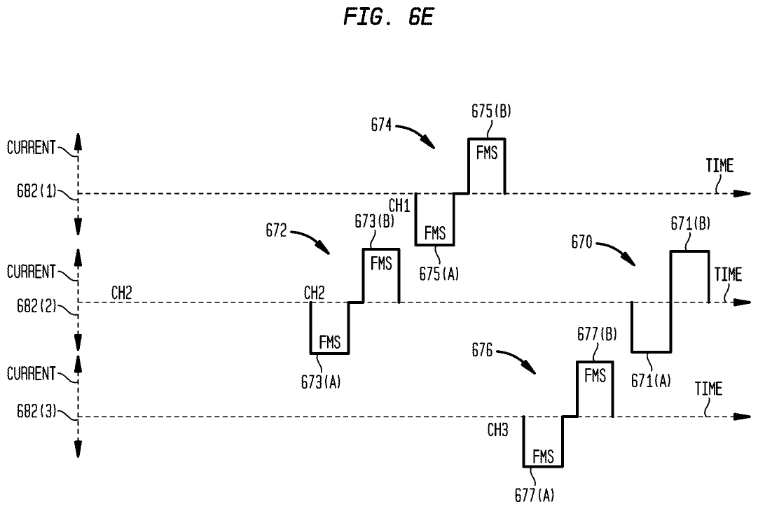

FIGS. 3, 5A, and 5B generally illustrate embodiments in which a facilitating signal and a stimulation signal, having different spatial extents, are delivered via the same stimulation channel (e.g., same electrode or group of electrodes). FIGS. 3 5A, and 5Balso illustrate embodiments in which there is a one-to-one correspondence between a facilitating signal and a stimulation signal. It is to be appreciated that such embodiments are merely illustrative and that facilitating signals and stimulation signals can be delivered in different combinations across different combinations of stimulation channels. FIGS. 6A, 6B, and 6C illustrate several such alternative embodiments.

Referring first to FIG. 6A, shown is an embodiment in which a facilitating signal 670 is delivered via stimulation channel (e.g., one or more electrodes) 682(2). Facilitating signal 670 is comprised of a first monophasic facilitating pulse 671(A) having a first polarity and a second monophasic facilitating pulse 671(B) having a second polarity that is opposite the first polarity. The facilitating pulse 671(A) is delivered with, for example, an intensity/level, timing, etc., to partially-depolarize an area of nerve cells (e.g., depolarize only to a level that is below the critical threshold of the nerve cells). That is, the facilitating pulse 671(A) is configured such that the nerve cells will not fire (e.g., will not undergo an action potential) in response to only the facilitating pulse 671(A).

FIG. 6A also illustrates that three biphasic stimulation signals, referred to as stimulation signals 672, 676, and 678, are delivered to subsets of the partially-depolarized nerve cells after delivery of the facilitating signal 670. In this example, stimulation signals 672 is delivered via the stimulation channel 682(2) (i.e., the same stimulation channel used to deliver facilitating signal 670), but stimulation signals 676 and 678 are delivered via different stimulation channels, referred to as stimulation channels 682(1) and 682(2), respectively.

As shown, stimulation signals 672 is comprised of a first stimulation pulse 673(A) having a first polarity, and a second stimulation pulse 673(B) having a second polarity that is opposite to the first polarity of first stimulation pulse 673(A). Similarly, stimulation signals 674 is comprised of a first stimulation pulse 675(A) having a first polarity, and a second stimulation pulse 675(B) having a second polarity that is opposite to the first polarity of first stimulation pulse 675(A). Finally, stimulation signals 676 is comprised of a first stimulation pulse 677(A) having a first polarity, and a second stimulation pulse 677(B) having a second polarity that is opposite to the first polarity of first stimulation pulse 677(A).

In accordance with the embodiments of FIG. 6A, the facilitating signal 670 is delivered to the nerves using an electrode configuration having an associated first spatial extent, while the stimulation signals 672, 674, and 676 are delivered to the nerves using one or more electrode configuration having associated spatial extents that are greater than the first spatial extent. Stated differently, the stimulation signals 672, 674, and 676 are each configured so as to result in a current spread that is less than the current spread resulting from the facilitating signal 670. The stimulation signals 672, 674, and 676 can be delivered using the same electrode configuration or using different electrode configurations with different spatial extents. That is, in certain embodiments, stimulation signal 672 could be delivered using one electrode configuration, while stimulation signals 674 and 676 are delivered using the same or different electrode configurations. However, as noted above, the electrode configurations used to deliver stimulation signals 672, 674, and 676 each have a greater spatial extent than the electrode configuration used to deliver facilitating signal 670. The relative differences in these spatial extents can be the result of different electrode configurations, such as monopolar stimulation, wide/defocused stimulation, focused (e.g., multipolar current focusing) stimulation, etc., as described, for example, with reference to FIGS. 4A-4C.

In the example of FIG. 6A, the facilitating pulse 671(A) is delivered with, for example, an intensity/level, timing, etc., to partially-depolarize an area of nerve cells (e.g., depolarize only to a level that is below the critical threshold of the nerve cells). That is, the facilitating pulse 671(A) is configured such that the nerve cells will not fire (e.g., will not undergo an action potential) in response to only the facilitating pulse 671(A). The area of partially-depolarized nerve cells, in response to the facilitating signal 670, is generally represented in FIG. 6B by dashed oval 684 with the horizontal lines In this example, facilitating pulse 671(B) is used for charge balancing.

As noted, the three stimulation signals 672, 674, and 676, are delivered to subsets of the partially-depolarized nerve cells 684 after delivery of the facilitating signal 670. In FIG. 6B, these subsets are represented by dashed ovals 685(2), 685(1), and 685(3) with vertical lines. The areas of nerve cells that fire/activate are represented in FIG. 6B by the intersections of the horizontal and vertical lines. That is, stimulation signal 672 is delivered to subset 685(2) of the partially-depolarize nerve cells 684, stimulation signal 674 is delivered to subset 685(2) of the partially-depolarize nerve cells 684, and stimulation signal 676 is delivered to subset 685(3) of the partially-depolarize nerve cells 684.

It is to be appreciated that the specific combination of facilitating signal and stimulation signals shown in FIG. 6A is merely illustrative and that, in accordance with embodiments presented herein, facilitating signals and stimulation signals could be delivered in various combinations.

For example, FIG. 6C illustrates an alternative embodiment that is similar to that of FIG. 6A, except that no stimulation signal is delivered on stimulation channel 682(2) (i.e., stimulation signal 672 is omitted). FIG. 6D illustrates an alternative embodiment that is similar to that of FIG. 6A, except that facilitating signal 670 is wrapped around the stimulation signals 672, 674, and 676. That is, in the embodiment of FIG. 6D, the monophasic facilitating pulse 671(A) is delivered prior to the biphasic stimulation signals 672, 674, and 676 to partially-depolarize the area of nerve cells 684. However, the facilitating pulse 671(B) is not delivered until after the biphasic stimulation signals 672, 674, and 676 for charging balancing of the facilitating pulse 671(A). While, this stimulus may raise other issues of time over which charge must be recovered, it illustrates that there are a variety of embodiments of facilitating stimuli. In another example, facilitating signals 670 could continue to be delivered during delivery of one or more of the biphasic stimulation signals 672, 674, and 676. FIG. 6E illustrates yet another example where the facilitating signal 670 is delivered after delivery of one or more of the biphasic stimulation signals 672, 674, and 676.

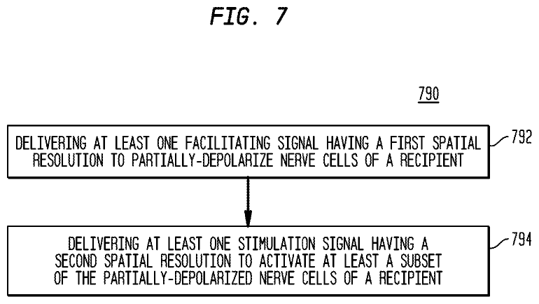

FIG. 7 is a flowchart of an example method 790, in accordance with embodiments presented herein. Method 790 begins at 792 where at least one facilitating signal having a first spatial extent is delivered to partially-depolarize nerve cells of a recipient. At 794, at least one stimulation signal having a second spatial extent is delivered to activate at least a subset of the partially-depolarized nerve cells of a recipient, where the second spatial extent is different from the first spatial extent.

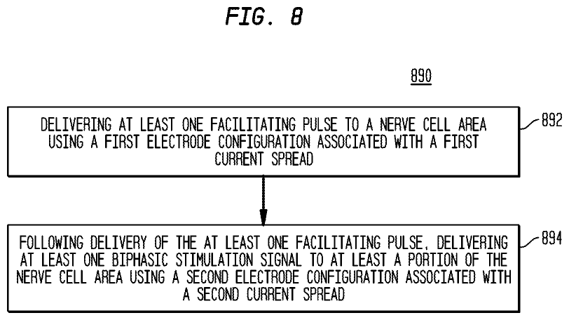

FIG. 8 is a flowchart of an example method 890, in accordance with embodiments presented herein. Method 890 begins at 892 where at least one facilitating pulse is delivered to a nerve cell area using a first electrode configuration associated with a first current spread. At 894, following delivery of the at least one facilitating pulse, at least one biphasic stimulation signal is delivered to at least a portion of the nerve cell area using a second electrode configuration associated with a second current spread that is different from the first current spread.

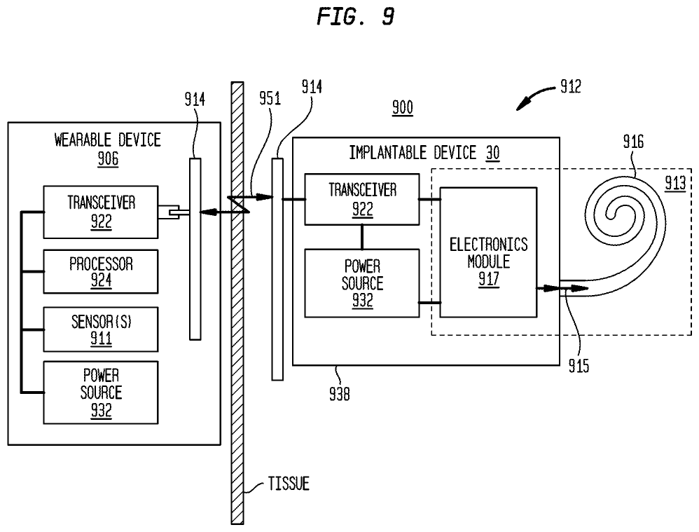

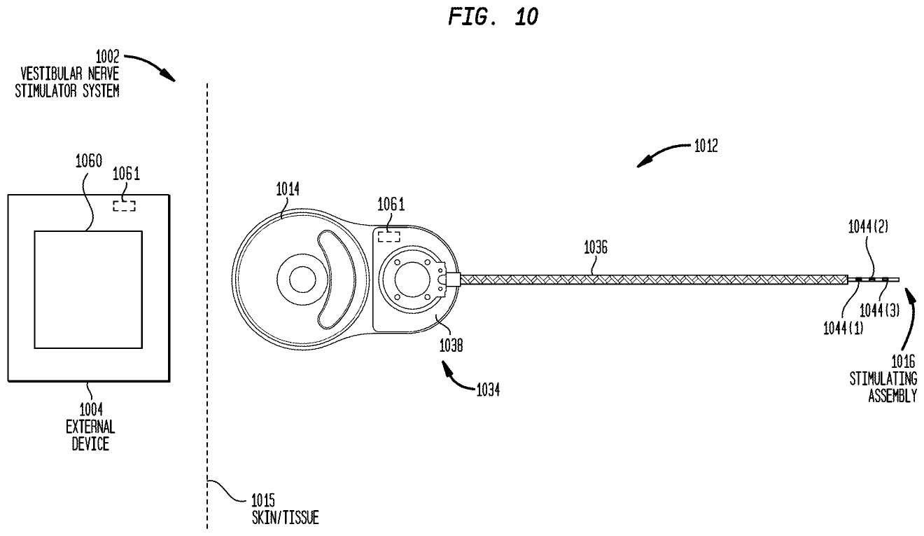

As previously described, the technology disclosed herein can be applied in any of a variety of circumstances and with a variety of different devices. For example, FIGS. 9-11 illustrate example devices configured to deliver facilitating signals and stimulation signals, as described above, in accordance with embodiments presented herein. As described further below, FIG. 9 illustrates an example implantable stimulation system, FIG. 9 illustrates an example vestibular stimulator, and FIG. 10 illustrates a retinal prosthesis. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 9 is a functional block diagram of an implantable stimulator system 900 that can benefit from the technologies described herein. The implantable stimulator system 900 includes the wearable device 906 acting as an external processor device and an implantable device 912 acting as an implanted stimulator device. In examples, the implantable device 912 is an implantable stimulator device configured to be implanted beneath a recipient's tissue (e.g., skin). In examples, the implantable device 912 includes a biocompatible implantable housing 938. Here, the wearable device 906 is configured to transcutaneously couple with the implantable device 912 via a wireless connection to provide additional functionality to the implantable device 912.

In the illustrated example, the wearable device 906 includes one or more sensors 911, a processor 924, a transceiver 922, and a power source 932. The one or more sensors 911 can be one or more units configured to produce data based on sensed activities. In an example where the stimulation system 900 is an auditory prosthesis system, the one or more sensors 911 include sound input sensors, such as a microphone, an electrical input for an FM hearing system, other components for receiving sound input, or combinations thereof. Where the stimulation system 900 is a visual prosthesis system, the one or more sensors 911 can include one or more cameras or other visual sensors. Where the stimulation system 900 is a cardiac stimulator, the one or more sensors 911 can include cardiac monitors. The processor 924 can be a component (e.g., a central processing unit) configured to control stimulation provided by the implantable device 912. The stimulation can be controlled based on data from the sensor 911, a stimulation schedule, or other data. Where the stimulation system 900 is an auditory prosthesis, the processor 924 can be configured to convert sound signals received from the sensor(s) 911 (e.g., acting as a sound input unit) into signals 951. The transceiver 922 is configured to send the signals 951 in the form of power signals, data signals, combinations thereof (e.g., by interleaving the signals), or other signals. The transceiver 922 can also be configured to receive power or data. Stimulation signals can be generated by the processor 924 and transmitted, using the transceiver 922, to the implantable device 912 for use in providing stimulation.

In the illustrated example, the implantable device 912 includes a transceiver 922, a power source 932, and a medical instrument 913 that includes an electronics module 917 and a stimulation arrangement 916. The electronics module 917 can include one or more other components to provide medical device functionality. In many examples, the electronics module 917 includes one or more components for receiving a signal and converting the signal into the stimulation signal 915. The electronics module 917 can further include a stimulator unit. The electronics module 917 can generate or control delivery of the stimulation signals 915 to the stimulation arrangement 916. In examples, the electronics module 917 includes one or more processors (e.g., central processing units or microcontrollers) coupled to memory components (e.g., flash memory) storing instructions that when executed cause performance of an operation. In examples, the electronics module 917 generates and monitors parameters associated with generating and delivering the stimulus (e.g., output voltage, output current, or line impedance). In examples, the electronics module 917 generates a telemetry signal (e.g., a data signal) that includes telemetry data. The electronics module 917 can send the telemetry signal to the wearable device 906 or store the telemetry signal in memory for later use or retrieval.

The stimulation arrangement 916 can be a component configured to provide stimulation to target tissue. In the illustrated example, the stimulation arrangement 916 is an electrode assembly that includes an array of electrode contacts disposed on a lead. The lead can be disposed proximate tissue to be stimulated. Where the system 900 is a cochlear implant system, the stimulation arrangement 916 can be inserted into the recipient's cochlea. The stimulation arrangement 916 can be configured to deliver stimulation signals 915 (e.g., electrical stimulation signals) generated by the electronics module 917 to the cochlea to cause the recipient to experience a hearing percept. In other examples, the stimulation arrangement 916 is a vibratory actuator disposed inside or outside of a housing of the implantable device 912 and configured to generate vibrations. The vibratory actuator receives the stimulation signals 915 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 922 can be components configured to transcutaneously receive and/or transmit a signal 951 (e.g., a power signal and/or a data signal). The transceivers 922 can be a collection of one or more components that form part of a transcutaneous energy or data transfer system to transfer the signal 951 between the wearable device 906 and the implantable device 912. Various types of signal transfer, such as electromagnetic, capacitive, and inductive transfer, can be used to usably receive or transmit the signal 951.

Each of the transceivers 922 can include or be electrically connected to a respective coil 914 for the transcutaneous transfer of power and/or data. The power sources 932 can be one or more components configured to provide operational power to other components. The power sources 932 can be or include one or more rechargeable batteries. Power for the batteries can be received from a source and stored in the battery. The power can then be distributed to the other components as needed for operation.

As should be appreciated, while particular components are described in conjunction with FIG. 9, technology disclosed herein can be applied in any of a variety of circumstances. The above discussion is not meant to suggest that the disclosed techniques are only suitable for implementation within systems akin to that illustrated in and described with respect to FIG. 9. In general, additional configurations can be used to practice the methods and systems herein and/or some aspects described can be excluded without departing from the methods and systems disclosed herein.

FIG. 10 illustrates an example vestibular stimulator system 1002, with which embodiments presented herein can be implemented. As shown, the vestibular stimulator system 1002 comprises an implantable component (vestibular stimulator) 1012 and an external device/component 1004 (e.g., external processing device, battery charger, remote control, etc.). The external device 1004 comprises a transceiver unit 1060. As such, the external device 1004 is configured to transfer data (and potentially power) to the vestibular stimulator 1012.

The vestibular stimulator 1012 comprises an implant body (main module) 1034, a lead region 1036, and a stimulating assembly 1016, all configured to be implanted under the skin/tissue (tissue) 1015 of the recipient. The implant body 1034 generally comprises a hermetically-sealed housing 1038 in which RF interface circuitry, one or more rechargeable batteries, one or more processors, and a stimulator unit are disposed. The implant body 134 also includes an internal/implantable coil 1014 that is generally external to the housing 1038, but which is connected to the transceiver via a hermetic feedthrough (not shown).

In certain embodiments, the external device 1004 and/or the vestibular stimulator 1012 can include one or more body motion sensors (e.g., accelerometers, gyroscopes, etc.) configured to capture motion signals associated with motion of the head or other parts of the recipient's body (e.g., capture angular accelerations of the head).

The stimulating assembly 1016 comprises a plurality of electrodes 1044(1)-(3) disposed in a carrier member (e.g., a flexible silicone body). In this specific example, the stimulating assembly 1016 comprises three (3) stimulation electrodes, referred to as stimulation electrodes 1044(1), 1044(2), and 1044(3). The stimulation electrodes 1044(1), 1044(2), and 1044(3) function as an electrical interface for delivery of electrical stimulation signals to the recipient's vestibular system.

The stimulating assembly 1016 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

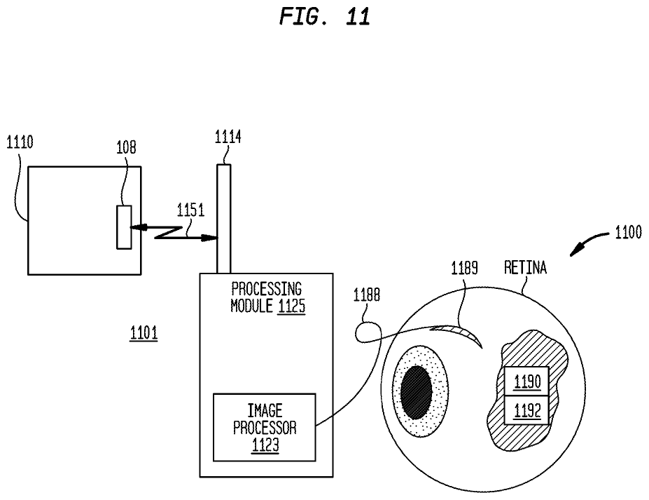

FIG. 11 illustrates a retinal prosthesis system 1101 that comprises an external device 1110 configured to communicate with a retinal prosthesis 1100 via signals 1151. The retinal prosthesis 1100 comprises an implanted processing module 1125 and a retinal prosthesis sensor-stimulator 1190 is positioned proximate the retina of a recipient. The external device 1110 and the processing module 1125 can communicate via coils 1108, 1114.

In an example, sensory inputs (e.g., photons entering the eye) are absorbed by a microelectronic array of the sensor-stimulator 1190 that is hybridized to a glass piece 1192 including, for example, an embedded array of microwires. The glass can have a curved surface that conforms to the inner radius of the retina. The sensor-stimulator 1190 can include a microelectronic imaging device that can be made of thin silicon containing integrated circuitry that convert the incident photons to an electronic charge.

The processing module 1125 includes an image processor 1123 that is in signal communication with the sensor-stimulator 1190 via, for example, a lead 1188 which extends through surgical incision 1189 formed in the eye wall. In other examples, processing module 1125 is in wireless communication with the sensor-stimulator 1190. The image processor 1123 processes the input into the sensor-stimulator 1190, and provides control signals back to the sensor-stimulator 1190 so the device can provide an output to the optic nerve. That said, in an alternate example, the processing is executed by a component proximate to, or integrated with, the sensor-stimulator 1190. The electric charge resulting from the conversion of the incident photons is converted to a proportional amount of electronic current which is input to a nearby retinal cell layer. The cells fire and a signal is sent to the optic nerve, thus inducing a sight perception.

The processing module 1125 can be implanted in the recipient and function by communicating with the external device 1110, such as a behind-the-ear unit, a pair of eyeglasses, etc. The external device 1110 can include an external light/image capture device (e.g., located in/on a behind-the-ear device or a pair of glasses, etc.), while, as noted above, in some examples, the sensor-stimulator 1190 captures light/images, which sensor-stimulator is implanted in the recipient.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method comprising:
delivering at least one facilitating signal having a first spatial extent to partially-depolarize nerve cells of a recipient; and
delivering at least one stimulation signal having a second spatial extent to activate at least a subset of the partially-depolarized nerve cells of a recipient,
wherein the second spatial extent is different from the first spatial extent.

2. The method of claim 1, wherein the second spatial extent is less than the first spatial extent.

3. The method of claim 1, wherein the second spatial extent is greater than the first spatial extent.

4. The method of claim 1, wherein the at least one facilitating signal comprises a first facilitating pulse having a first polarity and a second facilitating pulse having a second polarity.

5. The method of claim 4, wherein delivering the at least one facilitating signal having the first spatial extent to partially-depolarize the nerve cells of the recipient comprises:
delivering the first facilitating pulse and the second facilitating pulse prior to delivering the at least one stimulation signal.

6. The method of claim 4, wherein delivering the at least one facilitating signal having the first spatial extent to partially-depolarize the nerve cells of the recipient comprises:
delivering the first facilitating pulse and the second facilitating pulse after delivering the at least one stimulation signal.

7. The method of claim 4, wherein delivering the at least one facilitating signal having the first spatial extent to partially-depolarize the nerve cells of the recipient comprises:
delivering the first facilitating pulse prior to delivering the at least one stimulation signal; and
delivering the second facilitating pulse after delivering the at least one stimulation signal.

8. The method of claim 1, wherein delivering at least one facilitating signal having a first spatial extent to partially-depolarize nerve cells of a recipient comprises:
delivering the least one facilitating signal via a monopolar electrode configuration.

9. The method of claim 1, wherein delivering the at least one stimulation signal having the second spatial extent to activate at least the subset of the partially-depolarized nerve cells of a recipient comprises:
delivering the at least one stimulation signal using a multipolar electrode configuration.

10. The method of claim 1, wherein delivering the at least one stimulation signal having the second spatial extent to activate at least the subset of the partially-depolarized nerve cells of a recipient comprises:
delivering the at least one stimulation signal via a multipolar electrode configuration with a partial far-field return.

11. The method of claim 1, further comprising:
delivering the at least one facilitating signal and the at least one stimulation signal via a same stimulation channel.

12. The method of claim 1, further comprising:
delivering the at least one facilitating signal via a first stimulation channel; and
delivering the at least one stimulation signal via a second stimulation channel that is different from the first stimulation channel.

13. The method of claim 1, wherein delivering the at least one stimulation signal having a second spatial extent to activate at least a subset of the partially-depolarized nerve cells of a recipient, comprises:
delivering a plurality of stimulation signals to the recipient, wherein each of the stimulation signals has an associated spatial extent that is greater than a spatial extent of the first spatial extent.

14. The method of claim 13, wherein delivering the plurality of stimulation signals to the recipient comprises:
delivering two or more of the plurality of stimulation signals via different stimulation channels.

15. The method of claim 14, further comprising:
concurrently delivering the two or more of the plurality of stimulation signals.

16. The method of claim 13, wherein delivering the plurality of stimulation signals to the recipient comprises:
sequentially delivering two or more of the plurality of stimulation signals via a same stimulation channel.

17. The method of claim 1, wherein the least one facilitating signal has a first polarity, and wherein the at least one stimulation signal is at least one biphasic stimulation signal.

18-33. (canceled)

34. An implantable medical device, comprising:
one or more input devices configured to receive at least one input signal;
one or more processors configured to convert the at least one input signal to at least one output signal;
an electrode array configured to be implanted in a recipient; and
a stimulator unit configured to generate, based on the at least one output signal, at least one electrical stimulation signal and deliver, via the electrode array, the at least one electrical stimulation signal to a cell area of the recipient,
wherein, prior to delivery of the at least one electrical stimulation signal to the cell area, the stimulator unit is configured to generate and deliver, via the electrode array, at least a first facilitating pulse to the cell area, wherein the at least first facilitating pulse is delivered via a first electrode configuration and is configured to only partially-depolarize cells within the cell area.

35. The implantable medical device of claim 34, wherein the stimulator unit is configured to generate and deliver at least a second facilitating pulse to the cell area after delivery of the at least one electrical stimulation signal to the cell area.

36. The implantable medical device of claim 34, wherein the stimulator unit is configured to generate and deliver at least a second facilitating pulse to the cell area prior to delivery of the at least one electrical stimulation signal to the cell area.

37. The implantable medical device of claim 34, wherein the stimulator unit is configured to deliver the at least one electrical stimulation signal via a second electrode configuration that is different from the first electrode configuration.

38. The implantable medical device of claim 37, wherein the first electrode configuration is associated with a first current spread and the second electrode configuration is associated with a second current spread that is different from the first current spread.

39. (canceled)

40. (canceled)

41. The implantable medical device of claim 37, wherein the stimulator unit is configured to deliver the at least one facilitating pulse using a monopolar electrode configuration.

42. The implantable medical device of claim 41, wherein the stimulator unit is configured to deliver the at least one stimulation signal using a multipolar electrode configuration.

43. The implantable medical device of claim 41, wherein the stimulator unit is configured to deliver the at least one stimulation signal using a multipolar electrode configuration with a partial far-field return.

44. The implantable medical device of claim 37, wherein the stimulator unit is configured to deliver the at least one facilitating pulse signal and the at least one stimulation signal via a same stimulation channel.

45. The implantable medical device of claim 37, wherein the stimulator unit is configured to deliver the at least one facilitating pulse signal via a first stimulation channel and deliver the at least one stimulation signal via a second stimulation channel that is different from the first stimulation channel.

46. The implantable medical device of claim 34, wherein to deliver the at least one stimulation signal, the stimulator unit is configured to deliver a plurality of stimulation signals to the cell area wherein each of the stimulation signals is delivered using an electrode configuration associated with a current spread that is different from a current spread associated with the first electrode configuration.

47. The implantable medical device of claim 46, wherein the stimulator unit is configured to deliver two or more of the plurality of stimulation signals via different stimulation channels.

48. The implantable medical device of claim 47, wherein the stimulator unit is configured to concurrently deliver the two or more of the plurality of stimulation signals.

49. The implantable medical device of claim 46, wherein the stimulator unit is configured to sequentially deliver two or more of the plurality of stimulation signals via a same stimulation channel.

50-63. (canceled)
